(19) United States
(12) Patent Application Publication
(10) Pub. No.: US 2026/0286261 A1
(43) Pub. Date: Sep. 24, 2026

(54) SYSTEM AND METHOD FOR CULTIVATING ALGAE

(71) Applicant: SIGNIFY HOLDING B.V., EINDHOVEN (NL)

(72) Inventors: MARCELLINUS PETRUS CAROLUS MICHAEL KRIJN, EINDHOVEN (NL); MARC ANDRE DE SAMBER, LOMMEL (BE)

(21) Appl. No.: 19/472,732

(22) PCT Filed: Mar. 25, 2024
(86) PCT No.: PCT/EP2024/057928

§ 371 Date: Oct. 6, 2025

(30) Foreign Application Priority Data
Apr. 7, 2023 (EP) 23167206.4

(51) Int. Cl. C12M 1/00; C12M 1/26; C12M 1/34; C12M 1/36

(52) U.S. Cl. CPC C12M 21/02; C12M 33/00; C12M 41/06; C12M 41/12; C12M 41/32; C12M 41/34; C12M 41/36; C12M 41/48

(57) ABSTRACT

A system is disclosed for cultivating algae. The system comprises an irradiation system that is configured to irradiate to-be-harvested algae and an algae properties measurement system that is configured to measure one or more properties of the to-be-harvested algae. The system also comprises a data processing system that is configured to, based on the measured one or more properties of the to-be-harvested algae, determine a pre-harvest radiation recipe. The data processing system is further configured to cause the irradiation system to irradiate the to-be-harvested algae in accordance with the determined pre-harvest radiation recipe.

### TECHNICAL FIELD

This disclosure relates to a system and method for cultivating algae, in particular to such system wherein a pre-harvest radiation recipe is determined for to-be-harvested algae based on one or more measured properties of the to-be-harvested algae. This disclosure further relates to a computer-implemented method for irradiating to-be-harvested algae and to a computer program and computer-readable storage medium for performing such method.

### BACKGROUND

Algae (including microalgae) are microscopic organisms found in both seawater and freshwater. Like plants, (green) algae perform photosynthesis to increase their biomass. They are unicellular species which exist individually, or in chains or groups. Depending on the species, their sizes can range from a few micrometers to a few hundred micrometers. Unlike higher plants, algae do not have roots, stems, or leaves. Algae are rich in primary and secondary metabolites, such as vitamins, proteins, carotenoids, fatty acids and amino acids. The commercial value of the algae largely depends on the presence and concentration of these metabolites, and the creation of these substances can be influenced by radiation.

The global algae products market size is valued at USD 4.7 billion in 2021 and projected to reach USD 6.4 billion by 2026, implying a CAGR of 6.3% during this forecast period. One of the major factors that contribute to this growth in market size, is the increase in the consumption of algae-based nutraceuticals, fabrication of cosmetics, and generation of food supplements.

Algae easily absorb contaminants from the water. Because of this, for use in nutraceuticals, cosmetics, and food supplements, a controlled environment with clean water supply and uncontaminated fertilizers is required. Such a controlled environment is typically based on a flow-through photobioreactor. Such a photobioreactor is a closed system that allows natural radiation (daylight), and typically also artificial radiation, to enter the system to cultivate algae. The most used closed system is the so-called tubular photobioreactor, but other systems such bioreactors based on flat panels are also used.

In known horticulture systems for plants, all plants are typically planted at the same time, and hence, all plants are substantially in the same developmental stage (e.g., rooting, growing, flowering, fruit formation, and harvesting). Thus, the radiation can be adapted to the current developmental stage of the plants (e.g., rooting radiation, growing radiation, et cetera). By contrast, algae continuously multiply, leading to a mixture of plants of a large range of ages and hence development stage.

CN112680337A discloses a system for improving spirulina yield and nutritional content, comprising illuminating algae based on the growing stage and harvesting time of the algae. The system is similar to the horticultural systems described above, in which first growth light is applied to all algae in the bioreactor, after which pre-harvest light is applied to all algae in the bioreactor.

In light of the above, there is a need in the art for a system and method for cultivating algae that allows optimization of the metabolites in the algae, in particular in flow-through bioreactors.

US 2015/0299630 A1 discloses a photobioreactor for liquid cultures, US 2022/0312705 A1 discloses a culturing method and culturing device, and US 2018/0216056 A1 discloses a modular photobioreactors system for the cultivation of algae.

### SUMMARY

To that end, a system for cultivating algae is disclosed. The system comprises a growing compartment and a pre-harvest compartment, an output of the growing compartment being connected to an input of the pre-harvest compartment. The system further comprises a measurement system that is configured to measure one or more parameters that are relatable to a property of the to-be-harvested algae, and an irradiation system that is configured to irradiate to-be-harvested algae in the pre-harvest compartment. The system further comprises a data processing system that is configured to determine a pre-harvest radiation recipe, based on the measured one or more parameters, and to cause the irradiation system to irradiate the to-be-harvested algae in accordance with the determined pre-harvest radiation recipe.

The growing compartment and the pre-harvest compartment are typically spatially separated. For example, in the case of a tubular bioreactor, a first length of the tube may be referred to as the growing compartment, and a second length, downstream of the first length, may be referred to as the pre-harvest compartment. Similarly, for a flat-panel bioreactor, one or more first panels may be referred to as the growing compartment, and one or more second panels, downstream of the one or more first panels, may be referred to as the pre-harvest compartment. The connection between the output of the growing compartment and the input of the pre-harvest compartment may be direct or indirect. An example of an indirect connection is one in which there is an intermediate device or component positioned between the output of the growing compartment and the input of the pre-harvest compartment, e.g., a separator as described below.

Thus, the system can be a continuous-flow system, wherein the algae can be harvested more or less continuously and/or in batches that are small relative to the contents of the system. An advantage of such a system is that there is generally no interruption in the production, nor the need to finish/clean/start up the reactor again. If the harvesting occurs in batches, after harvesting of the algae (comprising, optionally, transferring the harvested algae to a post-treatment), the pre-harvest compartment can be filled/replenished again. In a substantially continuous system, there may be a substantially continuous flow from the growing compartment into the pre-harvest compartment and from the pre-harvest compartment to a harvesting system.

The measurement system may comprise one or more sensors configured to measure one or more parameters that are relatable to a property of the to-be-harvested algae. The parameters may be relatable to a property of the algae in a direct or indirect manner. For example, weather data such as solar irradiation data or air temperature data during the time period the to-be-harvested algae spend in the growing compartment, may be related to properties of the algae exiting the growing compartment (using, for instance, an analytical or statistical model). Another example is measurement of nutrients in the suspension that have not been absorbed by the algae. Other parameters can be related more directly to a property of the algae, such as a density or color of the algae (typically in a suspension). The measurement system may comprise a communication interface to enable communication with the data processing system.

The irradiation system may comprise at least one artificial radiation source. In a typical embodiment, the irradiation system comprises a plurality of controllable radiation sources, e.g., LEDs. The irradiation system may comprise a communication interface to enable communication with the data processing system.

The data processing system may comprise a processor and a memory, communicatively coupled to the processor. The memory may store computer-readable instructions which, when executed by the processor, configure the processor to perform one or more of the method steps described herein. To this end, the data processing system may be communicatively connected with the irradiation system, using, e.g., a wired or wireless connection. The data processing system may have a communication interface to allow communication with the measurement system and the irradiation system. Typically, the processor causes the data processing system to generate one or more control signals. The irradiation system is configured to receive the control signals and to irradiate algae as defined by the control signals.

The system may further comprise a harvesting system that is configured to harvest the to-be-harvested algae. The data processing system may be configured to cause the harvesting system to harvest the to-be-harvested algae after the to-be-harvested algae have been irradiated in accordance with the pre-harvest radiation recipe. The harvesting system may comprise a communication interface to enable communication with the data processing system.

It is noted that algae are different from typical crops (plants) in a number of aspects. For plants, all plants in a field or greenhouse typically follow the same crop cycle. They are planted at essentially the same time and are also harvested at the same time. In other words, the crop development is typically spatially homogeneous and temporally heterogeneous. Thus, for plants, a radiation recipe that is optimal for growth (which may vary over time) is applied to the entire crop throughout the plant's growth cycle, except for the last few days before harvest. During these last few days before harvest, a radiation treatment is used that specifically increases nutritional content of the crop. This is called a pre-harvest treatment. It is applied to plants that are in the same growth phase (plants that have the same age).

For algae, this is different. New algae keep forming throughout the growth period, and consequently, there is always a population of all ages. Especially for flow-through bioreactors, the applied radiation is typically temporally substantially homogeneous, and spatially heterogeneous, with different radiation recipes being applied to different parts of the bioreactor.

The pre-harvest radiation may promote generation of certain assimilates in the algae, and/or may inhibit generation of other assimilates. In general, the growing stage is used to optimize the amount of algae (defined by, e.g., biomass per unit volume or biomass per unit volume per unit time), whereas the pre-harvest stage is used to optimize the composition of the algae (e.g., concentration of carotenoids, flavonoids, vitamins, et cetera).

In an embodiment, at least one of the one or more parameters that are relatable to a property of the to-be-harvested algae is selected from:
an amount of irradiation received by the algae over a preceding period of time, and/or
a temperature of the algae over a preceding period of time.

The amount of irradiation may refer to natural irradiation (e.g., solar irradiation) and/or artificial irradiation (e.g., grow irradiation or supplemental irradiation as described below). More in general, the at least one parameter may be based on the measured or expected development of the algae in the growing compartment. The parameter may be implemented as a single number (or few numbers), e.g., an integral or an average or another statistical representation of a temporal evolution of a measured quantity, or as a time-series comprising many numbers.

In an embodiment, the to-be-harvested algae are suspended in a fluid, forming an algae suspension, and at least one of the one or more parameters that are relatable to a property of the to-be-harvested algae represents a property of the algae suspension. Said property of the algae suspension may be selected from:
a transparency or opacity of the algae suspension, possibly at one or more predetermined wavelengths,
a density of the algae in the algae suspension,
a colour of the suspension,
an amount or density of chlorophyll in the algae suspension,
an amount or density of one or more types of carotenoids in the algae suspension,
an amount or density of one or more types of flavonoids in the algae suspension,
a photosynthetic efficiency or stress level of the algae in the algae suspension,
an O₂ level of the algae suspension,
a CO₂ level of the algae suspension,
an acidity of the algae suspension,
a nutrient level of the algae suspension, and/or
a temperature of the algae suspension.

The density of the algae in the algae suspension may refer to, e.g., a numerical density (number of cells per unit volume), a mass density (algae biomass per unit volume), a volumetric density (volume of algae cells per unit volume), et cetera.

Some of these properties can be used both as a measure of a property of the algae and as input to determine parameters of the light recipe. For example, the transparency (or opacity) of the algae suspension can provide information about their chemical composition, size distribution, et cetera, and but can also inform a required light intensity to ensure all algae are illuminated with at least a certain minimum intensity.

In an embodiment, the algae are contained in an essentially transparent container, e.g., in the form of an algae suspension. In such an embodiment, the measurement system may comprise a radiation emitter positioned on a first side of the container and a radiation detector positioned on a second side of the container. The radiation detector may be configured to detect radiation transmitted through and/or reflected by the algae suspension.

The detected radiation may include fluorescence from the algae, invoked by radiation from the radiation source that is absorbed by the algae. The fluorescence signal may be used to derive information on the efficiency of the photosynthetic system of the algae (i.e., information of the efficiency of converting light into biomass). The fluorescence signal may also be used to determine whether the algae experience “stress”.

At least one of the one or more parameters may be determined based on the detected radiation.

The radiation detection may be single-wavelength, multispectral, or hyperspectral. The radiation may be optimized (e.g., through selection of one or more suitable wavelengths) to determine one or more properties of the algae suspension, e.g., as described above. The radiation may also be chosen such as to provide information on the interaction of the radiation to be used in the pre-harvest radiation recipe with the algae suspension. In an embodiment with supplemental radiation (as described below), the transmission and/or reflection of the supplemental radiation may be measured.

In an embodiment, the system comprises a water temperature control system that is configured to control the temperature of the algae suspension. The data processing system is configured to determine a temperature for the algae suspension. The determined temperature may be based on the determined pre-harvest radiation recipe, or the pre-harvest radiation recipe may be determined based on the determined temperature. The data processing system is furthermore configured to cause the water temperature control system to cause the algae suspension to have the determined temperature.

In general, the water temperature may be affected by the environment of the system, e.g., by the ambient temperature. This is particularly relevant for bioreactors that are placed outside. However, the ambient temperature is not necessarily the optimum temperature for the development of the algae (e.g., for photosynthesis), or not the optimum temperature for the current (natural and/or artificial) light conditions.

In some embodiments, the water temperature control system can only affect the water temperature in a single direction, e.g., up or down. Thus, the water temperature control system can comprise a heater and/or a cooler. Suitable heaters and coolers are well known in the art. The water temperature control system may further comprise a communication interface to enable communication with the data processing system.

The determined water temperature can be part of the pre-harvest radiation recipe. The determined water temperature can also be determined by a (different) algorithm, using the pre-harvest radiation recipe as input. This can be a simple table look-up, or a more complicated algorithm.

Alternatively, the water temperature may be determined based on other criteria, e.g., based on the measured one or more parameters that are relatable to a property of the to-be-harvested algae. In that case, the pre-harvest radiation recipe may be determined based on the determined water temperature.

In an embodiment, the irradiation system comprises one or more controllable radiation sources for generating artificial radiation and/or one or more controllable radiation filters and/or radiation concentrators.

The controllable radiation sources can be, e.g., LED radiation sources, HID radiation sources, laser radiation sources, or other suitable radiation sources. The one or more controllable radiation sources may include means for controlled interaction with the algae, e.g., through filtering and/or concentrating the generated radiation, and/or through modifying the flow rate of, and hence interaction time with, the algae. The radiation filters may be configured to selectively block, in part or in full, radiation of one or more predetermined wavelength regions. The radiation concentrators may be configured to concentrate radiation on the algae, e.g., using a lens or mirror. Filter and concentrators can also be combined, e.g., by wavelength conversion, reducing an intensity of radiation of a first wavelength (filtering) while increasing radiation of a second wavelength (concentrating).

The one or more radiation filters may have, for instance, controllable radiation filtering properties and/or a controllable position. Similarly, the one or more radiation concentrators may have, for instance, controllable radiation concentrating properties and/or a controllable position. The controllable position may allow the filters and/or concentrators to be selectively inserted and removed between a radiation source and the algae.

In an embodiment, the data processing system is further configured to determine a supplemental radiation recipe, and to cause the irradiation system to irradiate algae in the growing compartment in accordance with the determined supplemental radiation recipe. The determination of the pre-harvest radiation recipe may be based on the determined supplemental radiation recipe. The determination of the supplemental radiation recipe may be based on the measured one or more parameters that are relatable to a property of the to-be-harvested algae. Additionally, or alternatively, the determination of the supplemental radiation recipe may be based on measured or known environmental properties, such as length of day and/or expected or measured amount of natural radiation, or even external properties such as market forecasts.

The supplemental radiation may ensure the algae entering the pre-harvest compartment have certain predetermined characteristics and/or that the algae suspension entering the pre-harvest compartment has certain predetermined characteristics, e.g., a minimum density (this may indicate that the algae are sufficiently grown to be ready for the next phase). For example, if there has been a paucity of natural radiation (e.g., solar radiation) leading to a low algae growth, supplemental radiation may be used to promote algae growth and ensure a sufficient amount (concentration) of algae in the algae suspension. In this example, the supplemental radiation recipe may be determined based on measurements on the algae (suspension) or based on measurements on the amount of incoming natural radiation. This can be done at different time scales; for example, if essentially the entire growing compartment is irradiated by the supplemental radiation, the supplemental radiation recipe may be determined based on the, e.g., the instantaneous or diurnally received irradiation. On the other hand, if only a limited part of the growing compartment is irradiated by the supplemental radiation, the supplemental radiation recipe may be determined based on the naturally received irradiation over the period that the algae in the irradiated part were travelling through the non-irradiated part of the growing compartment.

The algae in the growing compartment may also be referred to as other algae than the to-be-harvested algae.

In an embodiment, the measurement system comprises one or more sensors in the growing compartment and/or at or close to the input of the pre-harvest compartment. The one or more sensors are arranged to measure at least one of the one or more parameters that are relatable to a property of the to-be-harvested algae.

Thus, the sensors may be arranged to measure at least one of the one or more parameters that are relatable to a property of the to-be-harvested algae before the irradiation of the to-be-harvested algae in accordance with the determined pre-harvest radiation recipe (and also, of course, before determination of the pre-harvest radiation recipe).

In some embodiments, the measurement system further comprises one or more additional sensors arranged to measure the at least one of the one or more parameters that are relatable to a property of the to-be-harvested algae after the irradiation of the to-be-harvested algae in accordance with the determined pre-harvest radiation recipe.

By measuring the same parameters before and after irradiation in accordance with the determined pre-harvest radiation recipe, the effect of the pre-harvest radiation recipe may be determined. This information may be used to optimize the pre-harvest radiation recipe.

In an embodiment, the system further comprises a separator, an input of the separator being connected to the output of the growing compartment, a first output of the separator being connected to an input of the growing compartment, and a second output of the separator being connected to the input of the pre-harvest compartment. The separator is configured to split the algae suspension received from the growing compartment into a first part and a second part, the first part being provided to the input of the growing compartment by the first output of the separator and the second part being provided to the input of the pre-harvest compartment by the second output of the separator. A ratio between the first part and the second part may be dependent on the measured one or more parameters and/or on the pre-harvest-radiation recipe or vice versa. The separator may also be referred to as a splitter.

By connecting an output of the separator to an input of the growing compartment, part of the algae may be recycled, such that the system may be used in a continuous manner, without the need to add new algae to the system.

If the ratio between the first and second parts is controllable, the amount (or fraction) of to-be-harvested algae may be controlled. For example, if the current pre-harvest radiation recipe needs a relatively long time, a smaller fraction of algae may be diverted to the pre-harvest compartment, whereas when the current pre-harvest radiation recipe needs a relatively short time, a larger fraction of algae may be diverted to the pre-harvest compartment.

Conversely, the pre-harvest radiation recipe may be based on the ratio between the first part and the second part. This way, the pre-harvest radiation recipe can be determined or adjusted based on the amount of to-be-harvested algae. Thus, a planning can be defined to have a predetermined harvest amount available at a preferred time with a preferred amount.

In an embodiment, the irradiation system is configured to selectively irradiate (only) the to-be-harvested algae in accordance with the pre-harvest radiation recipe. In an embodiment, the data processing system is configured to cause the irradiation system to selectively irradiate (only) the to-be-harvested algae in accordance with the pre-harvest radiation recipe.

This way, the other algae than the to-be-harvested algae, e.g., the algae in the growing compartment, are not adversely affected by the radiation of the pre-harvest radiation recipe.

In an embodiment, the pre-harvest radiation recipe defines, for a particular time period, one or more properties of radiation provided to the to-be-harvested algae, wherein the one or more properties of radiation comprise at least one of:
a photon flux of the radiation as generated by the irradiation system,
a photon flux density of the radiation as received by the to-be-harvested algae,
a spectral power distribution of the radiation generated by the irradiation system, and
a timing of the irradiation.

In such an embodiment, the data processing system is configured to cause the irradiation system to generate radiation such that the radiation has the photon flux and/or photon flux density and/or the spectral power distribution and/or the timing of the irradiation as defined by the pre-harvest radiation recipe.

The timing of the irradiation may include temporal variation of the photon flux (density) and/or spectral power distribution, and/or the duration of the irradiation. The photon flux may be defined, e.g., in μmol/m²/s, or in any other useful quantity.

The irradiation system may comprise a radiation source with a controllable intensity and/or spectral distribution of the generated radiation. For example, the radiation source may comprise a plurality of independently (or per type) controllable LEDs and/or lasers with different wavelengths and/or a broad-spectrum radiation source (such as a HID) with adjustable radiation filters.

The irradiation system may comprise a controller to control radiation sources in the irradiation system. Alternatively, radiation sources in the irradiation system may be controlled directly by the data processing system.

Alternatively, or additionally, a flow rate of the algae that pass the radiation sources may be controlled, affecting the duration that the algae are irradiated by a fixed radiation source, without necessarily adjusting the output of the irradiation system.

In an embodiment, the data processing system is configured to determine the pre-harvest radiation recipe based on the one or more parameters that are relatable to a property of the to-be-harvested algae using a model, wherein the model associates sets of properties of the to-be-harvested algae with pre-harvest radiation recipes.

The model may be a machine-learning model or may be obtainable by a machine-learning method. Thus, in an embodiment, the data processing system is configured to construct the model based on machine-learning training data, wherein the machine-learning training data associate a plurality of sets of one or more algae properties to respective pre-harvest radiation recipes.

The model may be based on a difference in the at least one of the one or more parameters that are relatable to a property of the to-be-harvested algae before and after the irradiation of the to-be-harvested algae in accordance with the determined pre-harvest radiation recipe.

To this end, the measurement system may comprise sensors for measuring the relevant properties before and after application of the pre-harvest recipe, as described above. This way, good results may be obtained even without, necessarily, a deep understanding of the underlying physical and/or biological principles. Additionally, by continuously updating the model, the determined pre-harvest radiation recipe may be gradually improved. Additionally, or alternatively, samples of the algae may be taken before and after application of the pre-harvest radiation recipe; analysis of these samples may similarly inform the definition or finetuning of the model. Additionally, or alternatively, analysis data of the harvested algae at different stages of the post-processing of the algae can be used as input data for the model.

In an aspect, the disclosure relates to a computer-implemented method for irradiating to-be-harvested algae. The method comprises receiving from a measurement system a signal indicative of one or more parameters that are relatable to a property of the to-be-harvested algae, and determining, based on the one or more parameters as indicated by the signal, a pre-harvest radiation recipe. The method may further comprise causing an irradiation system to irradiate the to-be-harvested algae in accordance with the determined pre-harvest radiation recipe.

Thus, a system as described above may be controlled using this method. The method may be executed, for example, by the data processing system described above.

One aspect of this disclosure relates to a computer comprising a computer readable storage medium having computer readable program code embodied therewith, and a processor, preferably a microprocessor, coupled to the computer readable storage medium, wherein responsive to executing the computer readable program code, the processor is configured to perform any of the methods disclosed herein.

One aspect of this disclosure relates to a computer program or suite of computer programs comprising at least one software code portion or a computer program product storing at least one software code portion, the software code portion, when run on a computer system, being configured for executing any of the methods disclosed herein.

One aspect of this disclosure relates to a non-transitory computer-readable storage medium storing at least one software code portion, the software code portion, when executed or processed by a computer, is configured to perform any of the methods disclosed herein.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products as claimed in embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products as claimed in various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing systems (e.g., to the existing control systems) or be stored upon manufacturing of these systems.

Elements and aspects discussed for or in relation with a particular embodiment may be suitably combined with elements and aspects of other embodiments, unless explicitly stated otherwise. Embodiments of the present invention will be further illustrated with reference to the attached drawings, which schematically will show embodiments as claimed in the invention. It will be understood that the present invention is not in any way restricted to these specific embodiments.

### BRIEF DESCRIPTION OF THE DRAWINGS

Aspects of the invention will be explained in greater detail by reference to exemplary embodiments shown in the drawings, in which:

FIGS. 1A-1C schematically illustrate systems for cultivating algae according to embodiments;

FIGS. 2A-2C schematically illustrate irradiation systems for cultivating algae according to embodiments;

FIG. 3 is a flow chart illustrating an embodiment of a method for cultivating algae;

FIG. 4 is a flow chart illustrating another embodiment of the method for cultivating algae; and

FIG. 5 illustrates a data processing system according to an embodiment.

### DETAILED DESCRIPTION OF THE DRAWINGS

In the figures, identical reference numbers indicate identical or similar elements.

Algae cultivars like Arthrospira platensis (Spirulina) and Chlorella vulgaris (Chlorella), are examples of cultivars being grown for human consumption. These algae need a dark period (typically a photoperiod of 18 hours with 6 hours of darkness). Optimum growth takes place at (water) temperatures between 25 and 30° C. A large part of the commercial value of algae is determined by their nutritional content, typically defined as the presence and/or concentration of certain primary and/or secondary metabolites. Since algae have lots of similarities with plants, their nutritional content can be steered by radiation in a way similar to plants. Radiation optimal for algae biomass growth does not necessarily result in an optimal nutritional content.

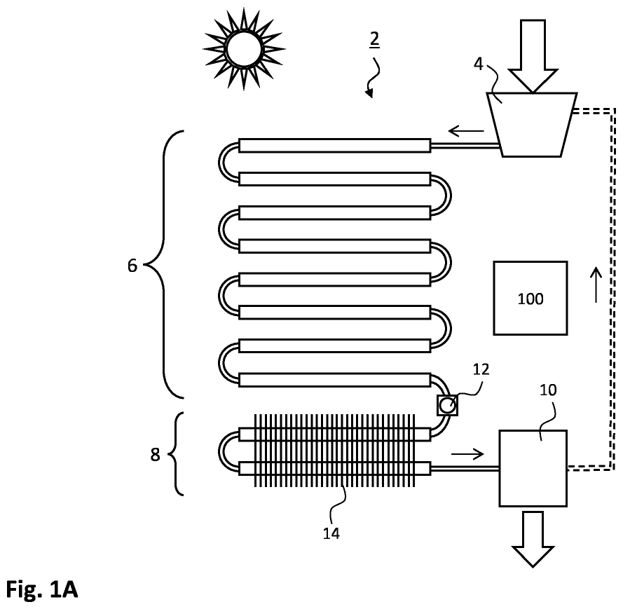

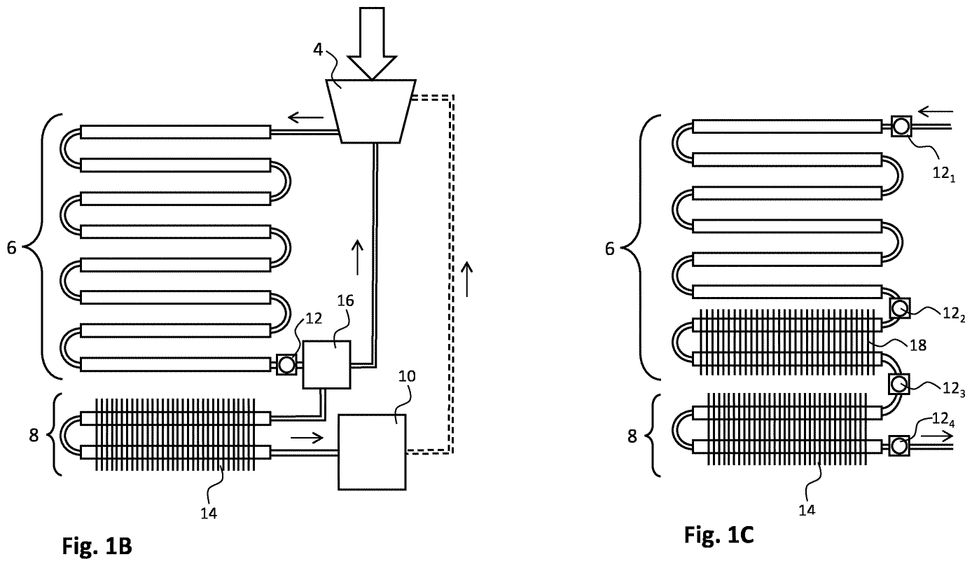

FIG. 1A schematically illustrates a system for cultivating algae according to a first embodiment. The system 2 comprises a feeding vessel 4 for adding algae, water and other ingredients to the system, such as nutrients and CO2. The feeding vessel 4 is connected, via a pipe, with an input of a growing compartment 6 of a bioreactor. Bioreactors for growing algae are well-known in the art. They typically comprise a plurality of interconnected containers, e.g., tubes or panels (sheets), through which a suspension containing the algae is transported. A bioreactor can also comprise only a single container, e.g., a single tube. The containers are typically transparent at the relevant wavelengths. For example, the containers are typically transparent to growing radiation. Growing radiation typically comprises a mixture of red and blue radiation. Red radiation may be understood as radiation having a wavelength between 600-750 nm, whereas blue radiation may be understood as radiation having a wavelength between 400-500 nm.

The bioreactor is often placed outside or inside a greenhouse, in order to absorb natural radiation. The lay-out of the bioreactor may be optimized to intercept a maximum amount of natural radiation which has a broadband spectrum.

An output of the growing compartment 6 is connected to an input of a pre-harvest compartment 8 of the bioreactor. The algae in the pre-harvest compartment may be referred to as to-be-harvested algae. The pre-harvest compartment 8 may be structurally similar or identical to the growing compartment 6; that is, depending on the implementation, the distinction between growing compartment 6 and pre-harvest compartment 8 may be functional rather than structural. An output of the pre-harvest compartment 8 is connected to a harvesting system 10. The harvesting system 10 is configured for harvesting the algae, e.g., using sedimentation, membrane separation, flocculation, flotation, or centrifugation. The fluid fraction that is left over after extraction of the algae may be provided to the feeding vessel to allow reuse of the fluid and of, e.g., nutrients still present in the fluid. The algae in the growing compartment 6 may also be referred to as ‘other algae than the to-be-harvested algae’ (i.e., algae in another stage or with a different composition, but still the same type of algae).

The system 2 further comprises a measurement system. The measurement system may comprise one or more sensors 12 configured to measure one or more parameters that are relatable to a property of the to-be-harvested algae. In the depicted example, sensor 12 is configured to measure one or more parameters of the algae suspension in between the growing compartment 6 and the pre-harvest compartment 8.

The sensor can be a radiation sensor and may comprise a radiation emitter and a radiation detector. The radiation emitter may be positioned on a first side of a container containing the algae suspension and a radiation detector positioned on a second side of the container. The radiation detector may be configured to detect radiation transmitted through and/or reflected by the algae suspension. At least one of the one or more parameters may be determined based on the detected radiation.

The radiation detection may be single-wavelength, multispectral, or hyperspectral, and may include the detection of fluorescence originating from the algae. The radiation may be optimized (e.g., through selection of one or more suitable wavelengths) to determine one or more properties of the algae suspension. The radiation may also be chosen such as to provide information on the interaction of the radiation to be used in the pre-harvest radiation recipe with the algae suspension (e.g., the absorption of blue radiation or UV radiation). In an embodiment with supplemental radiation (as described below), the transmission and/or reflection of the supplemental radiation may be measured.

In an embodiment, the measurement system comprises one or more sensors 12 in the growing compartment 6 and/or at or close to the input of the pre-harvest compartment 8. The one or more sensors are arranged to measure at least one of the one or more parameters that are relatable to a property of the to-be-harvested algae. Thus, the sensors 12 may be arranged to measure at least one of the one or more parameters that are relatable to a property of the to-be-harvested algae before the irradiation of the to-be-harvested algae in accordance with the pre-harvest radiation recipe is applied. The measurement system may comprise a processor to process input received from the sensors 12. The measurement system may comprise a communication interface to enable wired and/or wireless communication with data processing system 100.

For example, the radiation sensor 12 may be used to determine the amount of natural radiation (daylight) received (possibly per wavelength band) by the algae prior to arriving at the pre-harvest compartment. The pre-harvest radiation treatment can then be made dependent on the received amount of natural radiation prior to the pre-harvest radiation treatment. For example, in case the received amount of natural radiation is relatively low, the algae may not have produced a high nutritional content (since they have not been stressed by a high natural radiation level or high UV-A and UV-B level present in the natural radiation). In that case the pre-harvest radiation level may be increased to compensate. Next to the radiation level, also the radiation spectrum may be adapted, or the duration.

The optimal pre-harvest radiation treatment can depend on algae parameters such as the algae biomass density and the nutritional content. Such parameters can differ at different times and locations, depending on environmental parameters (such as water temperature, water glucose level, pH, electric conductivity, O₂ level (dissolved oxygen), CO₂ level (dissolved carbon dioxide), as well as natural radiation (daylight) level and, where applicable, supplemental radiation level as described below).

The irradiation system may comprise at least one radiation source 14. In a typical embodiment, the irradiation system comprises a plurality of radiation sources 14, typically controllable radiation sources, e.g., LEDs, for generating artificial radiation. The irradiation system may further comprise a controller. The controller may comprise a processor and a memory communicatively coupled to the processor. The irradiation system may comprise a communication interface to enable communication with data processing system 100. Additionally, or alternatively, the irradiation system may comprise one or more controllable radiation filters and/or radiation concentrators. Such filters and/or concentrators can be used, for example, to control aspects of an artificially generated radiation spectrum that cannot be sufficiently controlled by the radiation sources.

The irradiation system is configurable to a apply a pre-harvest radiation recipe to the algae in the pre-harvest compartment. (Immediately) prior to harvesting, the algae are given a pre-harvest radiation treatment, such as a radiation treatment aimed at providing stress to the algae plants, in order to invoke a response such as an increased production of nutritional compounds such as vitamins, proteins, carotenoids, fatty acids, amino acids vitamins, or a certain taste.

The controllable radiation sources can be, e.g., LED radiation sources, HID radiation sources, or other suitable radiation sources. The one or more controllable radiation sources may include a radiation source with controlled interaction with the algae, e.g., through filtering and/or concentrating the natural radiation, and/or through modifying the flow rate, and hence interaction time, of the algae. The radiation filters may be configured to selectively block, in part or in full, radiation of one or more predetermined wavelength regions. The radiation concentrators may be configured to concentrate radiation on the algae, e.g., using a lens or mirror. Filter and concentrators can also be combined, e.g., by wavelength conversion, reducing an intensity of radiation of a first wavelength (filtering) while increasing radiation of a second wavelength (concentrating).

The irradiation system may comprise a radiation source with a controllable intensity and/or spectral distribution of the generated radiation. For example, the radiation source may comprise a plurality of independently (or per type) controllable LEDs with different wavelengths and/or a broad-spectrum radiation source (such as a HID) with adjustable radiation filters.

The irradiation system may comprise a controller to control radiation sources in the irradiation system. Alternatively, radiation sources in the irradiation system may be controlled directly by the data processing system.

The one or more radiation filters may have, for instance, controllable radiation filtering properties and/or a controllable position. Similarly, the one or more radiation concentrators may have, for instance, controllable radiation concentrating properties and/or a controllable position. The controllable position may allow the filters and/or concentrators to be selectively inserted and removed between a radiation source and the algae.

The pre-harvest radiation offered is generally meant to stress the algae such that the algae will respond by producing the compounds of interest (e.g., nutritional content, colorants, taste compounds, et cetera). Such radiation typically contains a large fraction of blue radiation (e.g., about 450 nm), or UV-A radiation (e.g., 385-405 nm) and/or radiation levels in excess of 50 μmol m⁻² s⁻² (measured at the surface of the container). In some cases, UV-B radiation can also be applied beneficially (for which at least the relevant part of the container wall needs to be transparent for UV-B light). In general, algae may be sensitive both to absolute intensities of certain wavelengths and to relative intensities of different wavelengths.

In an embodiment, the system comprises a water temperature control system (not shown) that is configured to control the temperature of the algae suspension. In such an embodiment, data processing system 100 may be configured to determine a temperature for the algae suspension. The determined temperature may be based on the determined pre-harvest radiation recipe, or the pre-harvest radiation recipe may be determined based on the determined temperature. The data processing system 100 is furthermore configured to cause the water temperature control system to cause the algae suspension to have the determined temperature.

In some embodiments, the water temperature control system can only affect the water temperature in a single direction, e.g., up or down. Thus, the water temperature control system can comprise a heater and/or a cooler. Suitable heaters and coolers are well known in the art.

The determined water temperature can be part of the pre-harvest radiation recipe as temperature is also known to be a possible stressor for the algae and used solely or in combination or synergy with radiation as a stressor. The determined water temperature can also be determined by a (different) algorithm, using the pre-harvest radiation recipe as input. This can be a simple table look-up, or a more complicated algorithm.

Alternatively, the water temperature may be determined based on other criteria, e.g., based on the measured one or more parameters that are relatable to a property of the to-be-harvested algae. In that case, the pre-harvest radiation recipe may be determined based on the determined water temperature.

In general, the optimal water temperature depends on the radiation treatment. Typically, for optimal photosynthetic efficiency, a higher radiation level requires a higher water temperature. It can therefore be beneficial to adjust the water temperature in the pre-harvest compartment separately from the water temperature in other areas, to be able to invoke a stress response only at the location of the pre-harvest radiation. The system may further be configured to control one or more other relevant parameters in conjunction with the pre-harvest radiation recipe, such as, e.g., pH or CO₂ level of the algae suspension, et cetera.

The system 2 furthermore comprises a data processing system 100. The data processing system 100 is configured to determine a pre-harvest radiation recipe, based on the one or more parameters measured by the measurement system. The data processing system 100 is further configured to cause the irradiation system to irradiate the to-be-harvested algae in accordance with the determined pre-harvest radiation recipe. The data processing system 100 may also be configured to control the feeding vessel 4. An example of a data processing system 100 is described below with reference to FIG. 5.

The system may further comprise various other components that are commonly part of such bioreactors, such as a pump, valves, et cetera. These are not shown in the Figures. Some of these components may be controlled by the data processing system 100.

FIG. 1B shows a (tubular) system in which the algae on average can make several round trips before arriving at the location of the pre-harvest radiation treatment (not shown are the required pumps and valves at suitable locations). This allows regulating the relative amount of natural radiation and pre-harvest radiation received by the algae.

To that end, the system further comprises a separator 16. An input of the separator 16 is connected to the output of the growing compartment 6, and a first output of the separator 16 is connected to the input of the growing compartment 6. This connection can be direct or indirect; in the depicted example, the first output of the separator 16 is connected to an input of the feeding vessel 4, which is in turn connected to the growing compartment 4. A second output of the separator 16 is connected to the input of the pre-harvest compartment 8.

The separator 16 is configured to split the algae suspension received from the growing compartment 6 into a first part which is fed back into the growing compartment, and a second part which is fed into the pre-harvest compartment 8. The ratio between the first part and the second part may be fixed or adjustable. This way, part of the algae may be recycled, e.g., to serve as ‘seed’ for a next round, such that the system may be used in a continuous manner, without the need to add new algae to the system. Also, multiple rounds might be required to generate sufficient algae before any algae might be transferred into the pre-harvest system 8.

In other implementations, the system layout may be such that the algae pass the location of the pre-harvest radiation treatment more than once before being harvested. For example, harvesting system 10 may be configured to harvest only part of the algae, allowing another part of the algae to return to the growing compartment 6.

If the ratio between the first and second parts is controllable, this ratio can be made dependent on, e.g., the measured one or more parameters and/or on the pre-harvest-radiation recipe. The other way round is also possible: if, e.g., the ratio between the first and second parts is changed, the pre-harvest radiation recipe may be adjusted based on the changed ratio.

FIG. 1C shows only the bioreactor part of a system for cultivating algae. In this example, the irradiation system comprises, next to a pre-harvest radiation module 14, a supplemental radiation module 18 configured to provide supplemental radiation to the algae. The supplemental radiation is provided to algae in the growing compartment 6. Such supplemental radiation can be required during times that there is insufficient natural radiation, for example.

Often, the algae undergo natural radiation irradiation. Based on the amount of natural radiation received, supplemental radiation may be offered during times that the amount of natural radiation is insufficient. In case the algae growth takes place in an environment without natural radiation, the supplemental radiation can be substantially all radiation received by the algae in the growing compartment 6. Although in this figure, the supplemental radiation module is only shown over the final part of the growing compartment 6, in other examples, the supplemental radiation may be provided to a different part of the growing compartment 6, or to substantially the entire growing compartment 6.

In such an embodiment, the pre-harvest radiation recipe may take into account the natural radiation and supplemental radiation amount received by the algae in the growing compartment 6.

The data processing system may further be configured to determine a supplemental radiation recipe, and to cause the irradiation system 14,18 to irradiate algae in the growing compartment 6 in accordance with the determined supplemental radiation recipe. The determination of the pre-harvest radiation recipe may be based on the determined supplemental radiation recipe. The determination of the supplemental radiation recipe may be based on the measured one or more parameters that are relatable to a property of the to-be-harvested algae.

The depicted example comprises a plurality of sensors 12₁₋₄; a first sensor 12₁ is placed at the input of the growing compartment 6, a second sensor 12₂ is placed right before the supplemental radiation module 18, a third sensor 12₃ is placed between the growing compartment 6 and the pre-harvest compartment 8 (and consequently, between the supplemental radiation module 18 and the pre-harvest radiation module 14), and a fourth sensor 12₄ is placed at the output of the pre-harvest compartment. Of course, different arrangements are also possible, for example, a plurality of sensors covering essentially the entire growing compartment 6.

By measuring the same parameters before and after irradiation in accordance with the pre-harvest radiation recipe, the effect of the pre-harvest radiation recipe may be determined. Similarly, by measuring the same parameters before and after irradiation in accordance with the supplemental radiation recipe, the effect of the supplemental radiation recipe may be determined.

The supplemental radiation may ensure the algae entering the pre-harvest compartment 8 have certain predetermined characteristics and/or that the algae suspension entering the pre-harvest compartment 8 has certain predetermined characteristics, e.g., a minimum density. For example, if there has been a paucity of natural radiation (e.g., solar radiation) leading to a low algae growth, supplemental radiation may be provided by the irradiation system 18 to promote algae growth and to ensure a sufficient amount (concentration) of algae in the algae suspension.

The supplemental radiation recipe may be determined, for example, based on measurements on the algae (suspension), or based on measurements on the amount of incoming natural radiation. This can be done at different time scales; for example, if essentially the entire growing compartment 6 is irradiated by the supplemental radiation, the supplemental radiation recipe may be determined based on, e.g., the instantaneous or diurnally received irradiation. On the other hand, if only a limited part of the growing compartment 6 is irradiated by the supplemental radiation, the supplemental radiation recipe may be determined based on the naturally received irradiation over the period that the algae in the irradiated part were travelling through the non-irradiated part of the growing compartment 6.

In some embodiments, the measurement system further comprises one or more additional sensors arranged to measure the at least one of the one or more parameters that are relatable to a property of the to-be-harvested algae after the irradiation of the to-be-harvested algae in accordance with the determined pre-harvest radiation recipe.

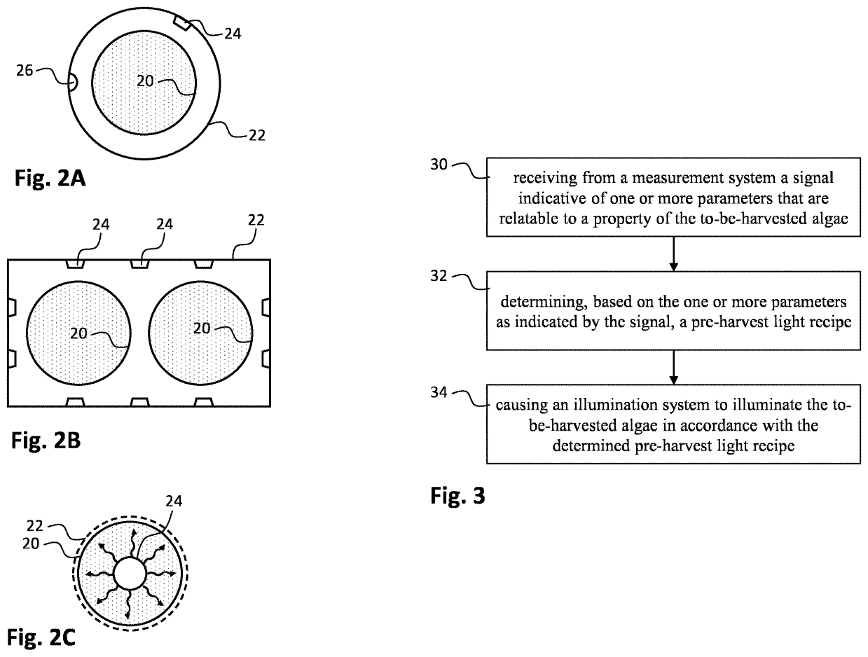

FIGS. 2A-2C schematically illustrate irradiation systems for cultivating algae according to embodiments.

In particular, FIG. 2A shows a cross-section through an irradiation system for a tubular bioreactor. At the location of the pre-harvest radiation treatment, tube 20, containing the algae, is shielded from the natural radiation (daylight) by surrounding the tube with a material 22 with high reflectivity on the inner side. Furthermore, a radiation source 24, e.g., a (linear) LED radiation module, may be used to irradiate the algae. A sensor 26 can be used to monitor absorption of the radiation provide by radiation source 24 by the algae, or to measure other parameters relatable to a property of the to-be-harvested algae. Although only a single radiation source 24 is shown, other embodiments may comprise a plurality of radiation sources.

With such a geometry, efficient use is made of the applied radiation, since almost all the radiation will be absorbed by the algae. Furthermore, shielding from natural radiation allows a large degree of control over the radiation received by the algae in tube 20. Moreover, the shielding prevents radiation from leaking out and reaching other algae than to-be-harvested algae, e.g., the algae in the growing compartment. Thus, the irradiation system can be configured to selectively irradiate (only) the to-be-harvested algae in accordance with the pre-harvest radiation recipe.

FIG. 2B shows a cross-section wherein a plurality of tubes 20 containing algae is shielded collectively with reflective material 22. A plurality of radiation sources 24 is shown, but the irradiation system may also comprise more or fewer radiation sources. A collective shielding may allow for a higher homogeneity of the applied radiation using fewer radiation sources. On the other hand, an individual shielding as shown in FIG. 2A allows for application of a spatially varying radiation recipe.

If the irradiation system is used to apply supplemental radiation, the shielding material 22 may be fully or partially transparent, in order to allow natural radiation to enter the irradiation system.

FIG. 2C shows a cross section through an irradiation system where the radiation source 24 is comprised in an inner tube which is surrounded by the tube 20 containing the algae. The irradiation system may optionally comprise a shielding material 22. Such an arrangement is particularly advantageous to apply a supplemental radiation recipe, as the artificial radiation is applied from the center, allowing essentially unobstructed entry of natural radiation from the outside (in an embodiment in which case material 22 is not shielding the natural radiation). It may also be used to provide radiation that cannot penetrate the walls of the tube 20; this can be particularly the case for UV radiation. As the inner tube 24 is shielded from outside influences by the outer tube 20, it may be made to different specifications, allowing different material choices.

In an embodiment, the irradiation system may be arranged to irradiate both (parts of) the growing compartment and the pre-harvest compartment. In that case, the data processing system may be configured to cause the irradiation system to selectively irradiate (only) the to-be-harvested algae in accordance with the pre-harvest radiation recipe, and/or to selectively irradiate (only) the other algae than the to-be-harvested algae in accordance with the supplemental radiation recipe. This way, the other algae than the to-be-harvested algae, e.g., the algae in the growing compartment, are not adversely affected by the radiation of the pre-harvest radiation recipe, and vice versa.

FIG. 3 is a flow chart illustrating an embodiment of a method for cultivating algae. The method may be executed, for example, by a data processing system 100 as described above with reference to FIG. 1A.

A step 30 comprises receiving, from a measurement system, a signal indicative of one or more parameters that are relatable to a property of the to-be-harvested algae. The one or more parameters may be obtained, for example, by means of suitable sensors 12, as described above with reference to FIG. 1A-1C.

The parameter measured by sensor 12 can be one of: a transparency or opacity of the algae suspension, a density of the algae in the algae suspension, a color of the algae suspension, an amount or density of chlorophyll in the algae suspension, an amount or density of one or more types of carotenoids in the algae suspension, an amount or density of one or more types of flavonoids in the algae suspension, a photosynthetic efficiency or stress level of the algae in the algae suspension, an O₂ level of the algae suspension, a CO₂ level of the algae suspension, an acidity of the algae suspension, a nutrient level of the algae suspension, a temperature of the algae suspension, and/or an amount of irradiation received by the algae suspension over a preceding period of time. Several sensors may be used to measure various of these properties. The parameter may be determined using one or more suitable sensors, e.g., an optical sensor, for example a sensor configured to determine fluorescence invoked by radiation from a natural or artificial radiation source.

A step 32 comprises determining, based on the one or more parameters as indicated by the signal, a pre-harvest radiation recipe. The pre-harvest radiation recipe may define, for a particular time period, one or more properties of radiation to be provided to the to-be-harvested algae. The one or more properties of the radiation may comprise at least one of:
a photon flux of the radiation as generated by the irradiation system,
a photon flux density of the radiation as received by the to-be-harvested algae,
a spectral power distribution of the radiation generated by the irradiation system, and
a timing of the irradiation.

An optional step 34 comprises causing an irradiation system to irradiate the to-be-harvested algae in accordance with the determined pre-harvest radiation recipe. For example, the data processing system may be configured to cause the irradiation system to generate radiation such that the radiation has the photon flux and/or photon flux density and/or the spectral power distribution and/or the timing of the irradiation as defined by the pre-harvest radiation recipe. The timing of the irradiation may include temporal variation of the photon flux (density) and/or spectral power distribution, and/or the duration of the irradiation.

Alternatively, or additionally, a flow rate of the algae that pass the radiation sources may be controlled, affecting the duration that the algae are irradiated by a possibly fixed radiation source, without necessarily adjusting the output of the irradiation system proper.

As an example, a method is described how to use sensors to determine a pre-harvest treatment to arrive at a desired carotenoid content.

At a location prior to where the algae receive the pre-harvest radiation treatment, the so-called chlorophyl-carotenoid index (CCI) is measured, for example by sensor 123 in FIG. 1C. It is known that the CCI is a measure of both the chlorophyl and carotenoid content (they are highly correlated). It involves measuring the reflection of the algae suspension at wavelengths of 532 nm and 630 nm. The (reflective) CCI is defined as

$$CCI = ( {R}_{532} - {R}_{630} ) / ( {R}_{532} + {R}_{630} ) ,$$

wherein R₅₃₂ denotes the reflectivity at 532 nm and R₆₃₀ denotes the reflectivity at 630 nm; a similar definition can be used for transmission values instead of reflection.

At a location where the algae have passed the pre-harvest radiation treatment location, the CCI is again measured, for example by sensor 12₄ in FIG. 1C.

Let CCI_{before} and CCI_{after} denote the CCI measurements at locations before (sensor 12₃) and after (sensor 12₄) the pre-harvest radiation treatment location. Also, let CCI_{target} be the desired (target) CCI at harvest. The aim is to have a pre-harvest radiation treatment that result as much as possible in CCI_{target}.

In case CCI_{before} is relatively low, a large pre-harvest radiation stress is applied (e.g., a high radiation intensity and/or a high blue or UV fraction). In case CCI_{before} is already high, only a moderate pre-harvest radiation stress needs to be applied (e.g., a low radiation intensity and/or a low blue or UV fraction). By applying different pre-harvest radiation treatments (i.e., different duration, spectrum, radiation level), by using machine learning, a system can gradually learn what pre-harvest radiation treatment to apply to, given a measured CCI_{before}, end up with CCI_{target}. In such a machine learning approach, next to CCI, other parameters can be taken into account, such as the aforementioned algae parameters and environmental parameters.

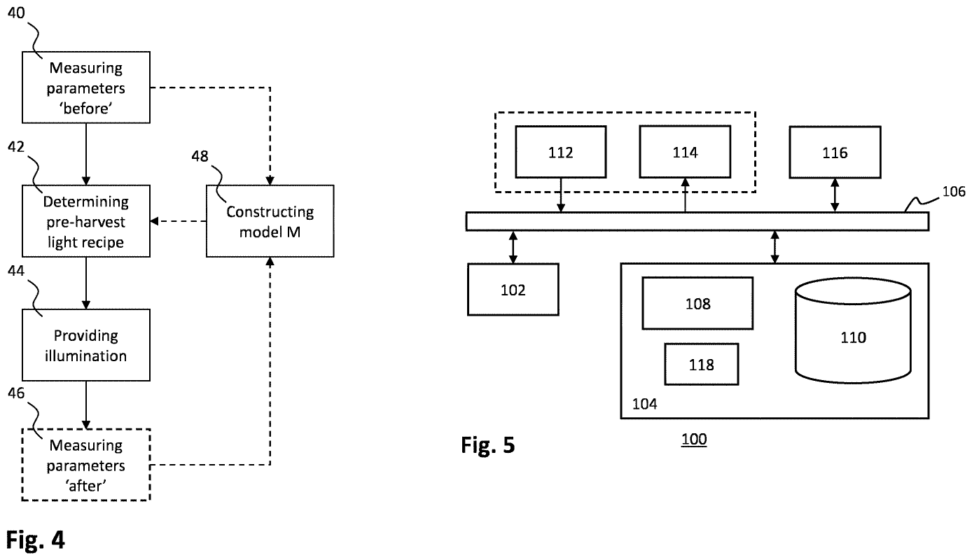

FIG. 4 is a flow chart illustrating another embodiment of the method for cultivating algae. Herein, a step 40 comprises measuring a parameter that is relatable to a property of the to-be-harvested algae. At least one of these algae parameters and environmental parameters may be quantified prior to providing the pre-harvest radiation treatment. This step may for example be embodied as reading out one or more sensors 12 of the measurement system as shown in the embodiment of FIG. 1A-1C, and optional data processing of the sensor readings. For example, the sensors may output a signal that is indicative of the reflected radiation at 532 nm and 640 nm (the measured parameters), which may be used to compute the CCI, which can in turn be linked to a chlorophyl and carotenoid content of the algae suspension. Thus, various analytical or modelling steps may be used to convert measured parameters into properties of the to-be-harvested algae.

Step 42 comprises, based on the measured one or more properties of the to-be-harvested algae, determining a pre-harvest radiation recipe. Then, step 44 comprises causing an irradiation system to irradiate the to-be-harvested algae in accordance with the determined pre-harvest radiation recipe. Step 46 comprises measuring an effectiveness of the pre-harvest radiation recipe, for example as described above.

A step 48 comprises constructing a model “M”, e.g., a machine-learning model, to associate a change in measured parameters and/or algae properties derived therefrom, with a pre-harvest radiation recipe.

In an embodiment, the data processing system is configured to determine the pre-harvest radiation recipe based on the one or more parameters that are relatable to a property of the to-be-harvested algae using a model M, wherein the model associates sets of properties of the to-be-harvested algae with pre-harvest radiation recipes.

In an embodiment, the data processing system is configured to construct the model based on machine-learning training data, wherein the machine-learning training data associate a plurality of sets of one or more measured parameters and/or one or more algae properties to respective pre-harvest radiation recipes. Thus, the model may be a machine-learning model or may be obtainable by a machine-learning method.

The model may be based on a difference in the at least one of the one or more parameters that are relatable to a property of the to-be-harvested algae before and after the irradiation of the to-be-harvested algae in accordance with the determined pre-harvest radiation recipe. To this end, the measurement system may comprise sensors for measuring the relevant properties before and after application of the pre-harvest radiation recipe, as described above.

Further, FIG. 4 illustrates that a single model M may be used for determining an appropriate pre-harvest radiation recipe based on a measured parameter. Model M may also be constructed, e.g., using machine learning methods known in the art. Model M may be understood to associate respective measured parameters of to-be-harvested algae on one side with respective pre-harvest radiation recipes on the other side. Thus, the model allows to determine, given a parameter of to-be-harvested algae, an appropriate pre-harvest radiation recipe for those to-be-harvested algae.

As indicated, model M may be constructed using machine learning methods known in the art. Such machine learning methods typically use training data for model construction. For example, for each of a plurality of batches of to-be-harvested algae, one or more parameters of the algae in question are measured, an effectiveness of the pre-harvest radiation recipe that has been applied to the batch in question is measured. The latter step may be performed by taking a sample of the batch before and after the pre-harvest radiation recipe has been applied and performing laboratory tests in order to determine the nutritional content increase of the algae during the application of the pre-harvest radiation recipe. Also, additional data from post-harvest analysis such as, e.g., the nutritional content, the productivity, etc., can be input for the model. Then, for each batch of to-be-harvested algae, its measured parameter and the pre-harvest radiation recipe that is applied and the effectiveness of the pre-harvest radiation recipe may be input into a system in association with each other. Herewith the training data for constructing model M are obtained.

FIG. 4 also shows that model M may be continuously refined. The parameter that is measured in step 40 may be stored in association with the effectiveness of the pre-harvest radiation recipe and in association with the pre-harvest radiation recipe itself. As such, these data may be used to enrich the training data and/or to improve model M.

FIG. 5 depicts a block diagram illustrating a data processing system as claimed in an embodiment.

As shown in FIG. 5, the data processing system 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the data processing system 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, a keyboard, a pointing device such as a mouse, a touch-sensitive display, an external control system referred to herein, or the like. Examples of output devices may include, but are not limited to, a monitor or a display, speakers, the LED driver, or the like. Input and/or output devices may be coupled to the data processing system either directly or through intervening I/O controllers.

In an embodiment, the input and the output devices may be implemented as a combined input/output device (illustrated in FIG. 5 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as, e.g., a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 100, and a data transmitter for transmitting data from the data processing system 100 to said systems, devices and/or networks. Modems, cable modems, and Ethernet cards are examples of different types of network adapter that may be used with the data processing system 100.

As pictured in FIG. 5, the memory elements 104 may store an application 118. In various embodiments, the application 118 may be stored in the local memory 108, the one or more bulk storage devices 110, or apart from the local memory and the bulk storage devices. It should be appreciated that the data processing system 100 may further execute an operating system (not shown in FIG. 5) that can facilitate execution of the application 118.

The application 118, being implemented in the form of executable program code, can be executed by the data processing system 100, e.g., by the processor 102. Responsive to executing the application, the data processing system 100 may be configured to perform one or more operations or method steps described herein.

In one aspect of the present invention, the data processing system 100 may represent a control system of a LED driver as described herein.

In another aspect, the data processing system 100 may represent a client data processing system. In that case, the application 118 may represent a client application that, when executed, configures the data processing system 100 to perform the various functions described herein with reference to a “client”. Examples of a client can include, but are not limited to, a personal computer, a portable computer, a mobile phone, or the like.

In yet another aspect, the data processing system 100 may represent a server. For example, the data processing system may represent an (HTTP) server, in which case the application 118, when executed, may configure the data processing system to perform (HTTP) server operations.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration but is not intended to be exhaustive or limited to the implementations in the form disclosed. Many modifications and variations will be apparent to those of ordinary skill in the art without departing from the scope and spirit of the present invention. The embodiments were chosen and described in order to best explain the principles and some practical applications of the present invention, and to enable others of ordinary skill in the art to understand the present invention for various embodiments with various modifications as are suited to the particular use contemplated.

1. A system for cultivating algae, the system comprising:
a growing compartment and a harvesting compartment, an output of the growing compartment being connected to an input of the harvesting compartment, and the harvesting compartment configured to, in operation, comprise to-be-harvested algae, wherein the to-be-harvested algae are suspended in a fluid, forming an algae suspension;
a measurement system for measuring one or more parameters that are relatable to a property of the to-be-harvested algae and generating a signal indicative of the one or more parameters, wherein at least one of the one or more parameters that are relatable to a property of the to-be-harvested algae represents a property of the algae suspension, said property of the algae suspension being selected from: a transparency or opacity of the algae suspension, a density of the algae in the algae suspension, a color of the algae suspension, an amount or density of chlorophyll in the algae suspension, an amount or density of one or more types of carotenoids in the algae suspension, an amount or density of one or more types of flavonoids in the algae suspension, a photosynthetic efficiency or stress level of the algae in the algae suspension, an O₂ level of the algae suspension, a CO₂ level of the algae suspension, an acidity of the algae suspension, a nutrient level of the algae suspension, and/or a temperature of the algae suspension;
an irradiation system for irradiating to-be-harvested algae in the harvesting compartment based on oner more control signals; and
a data processing system comprising a processor configured to:
receiving from the measurement system the signal indicative of one or more parameters that are relatable to a property of the to-be-harvested algae,
determine a pre-harvest radiation recipe, based on the one or more parameters as indicated in the signal, wherein the pre-harvest radiation recipe is configured to provide radiation comprising a large fraction of blue radiation or UV-A radiation or UV-B radiation,
generate one or more control signals for the irradiation system to irradiate the to-be-harvested algae in accordance with the determined pre-harvest radiation recipe, and
send the one or more control signals to the irradiation system.

2. The system as claimed in claim 1, wherein at least one of the one or more parameters that are relatable to a property of the to-be-harvested algae is based on an amount of irradiation received by the algae over a preceding period of time.

3. (canceled)

4. The system as claimed in claim 1, wherein
the algae suspension is contained in an essentially transparent container, wherein
the measurement system comprises a radiation emitter positioned on a first side of the container and a radiation detector positioned on a second side of the container, wherein, the radiation detector is configured to detect radiation transmitted through and/or reflected by the algae suspension; and wherein
the at least one of the one or more parameters is determined based on the detected radiation.

5. The system as claimed in claim 1, wherein the system comprises:
a water temperature control system that is configured to control the temperature of the algae suspension, wherein
the data processing system is configured to determine a temperature for the algae suspension and to cause the water temperature control system to cause the algae suspension to have the determined temperature, wherein the determined temperature is based on the determined pre-harvest radiation recipe, or wherein the pre-harvest radiation recipe is determined based on the determined temperature.

6. The system as claimed in claim 1, wherein the irradiation system comprises one or more controllable radiation sources, and/or
wherein the irradiation system comprises one or more controllable radiation filters and/or radiation concentrators.

7. The system as claimed in claim 1, wherein the data processing system is further configured to:
determine a supplemental radiation recipe, based on the measured one or more parameters, and
cause the irradiation system to irradiate algae in the growing compartment in accordance with the determined supplemental radiation recipe,
wherein the determination of the pre-harvest radiation recipe is further based on the determined supplemental radiation recipe.

8. The system as claimed in claim 1, wherein the measurement system comprises one or more sensors in the growing compartment and/or at or close to the input of the harvesting compartment, the one or more sensors being arranged to measure at least one of the one or more parameters that are relatable to a property of the to-be-harvested algae.

9. The system as claimed in claim 1, wherein the system further comprises a separator, an input of the separator being connected to the output of the growing compartment, a first output of the separator being connected to an input of the growing compartment, and a second output of the separator being connected to the input of the harvesting compartment;
wherein the separator is configured to split the algae suspension received from the growing compartment into a first part and a second part, the first part being provided to the input of the growing compartment by the first output of the separator and the second part being provided to the input of the harvesting compartment by the second output of the separator; and
wherein a ratio between the first part and the second part is dependent on the measured one or more parameters and/or on the pre-harvest-radiation recipe.

10. The system as claimed in claim 1, wherein
the irradiation system is configured to selectively irradiate only the to-be-harvested algae in accordance with the pre-harvest radiation recipe, and/or wherein
the data processing system is configured to cause the irradiation system to selectively irradiate only the to-be-harvested algae in accordance with the pre-harvest radiation recipe.

11. The system as claimed in claim 1, wherein the pre-harvest radiation recipe defines, for a particular time period, one or more properties of radiation provided to the to-be-harvested algae, wherein the one or more properties of radiation comprise at least one of:
a photon flux of the radiation as generated by the irradiation system,
a photon flux density of the radiation as received by the to-be-harvested algae,
a spectral power distribution of the radiation generated by the irradiation system, and
a timing of the irradiation; and
wherein the data processing system is configured to cause the irradiation system to generate radiation such that the radiation has the photon flux and/or photon flux density and/or the spectral power distribution and/or the timing of the irradiation as defined by the pre-harvest radiation recipe.

12. The system as claimed in claim 1, wherein
the data processing system is configured to determine the pre-harvest radiation recipe based on the one or more parameters that are relatable to a property of the to-be-harvested algae using a model, wherein the model associates sets of properties of the to-be-harvested algae with pre-harvest radiation recipes.

13. A method of irradiating to-be-harvested algae comprised in a harvesting compartment of a system for cultivating algae, wherein the to-be-harvested algae are suspended in a fluid, formed an algae suspension, the method comprising:
measuring by a measurement system one or more parameters that are relatable to a property of the to-be-harvested algae, wherein at least one of the one or more parameters that are relatable to a property of the to-be-harvested algae represents a property of the algae suspension, said property of the algae suspension being selected from: a transparency or opacity of the algae suspension, a density of the algae in the algae suspension, a color of the algae suspension, an amount or density of chlorophyll in the algae suspension, an amount or density of one or more types of carotenoids in the algae suspension, an amount or density of one or more types of flavonoids in the algae suspension, a photosynthetic efficienty or stress level of the algae in the algae suspension, an O₂ level of the algae suspension, a CO₂ level of the algae suspension, an acidity of the algae suspension, a nutrient level of the algae suspension, and/or a temperature of the algae suspension,
receiving from the measurement system a signal indicative of the one or more parameters that are relatable to a property of the to-be-harvested algae,
determining, based on the one or more parameters as indicated by the signal, a pre-harvest radiation recipe, wherein the pre-harvest radiation recipe is configured to provide radiation comprising a large fraction of blue radiation or UV-A radiation or UV-B radiation,
generating one or more control signals for an irradiation system to irradiate the to-be-harvested algae in accordance with the determined pre-harvest radiation recipe,
sending the one or more control signals to the irradiation system, and
irradiating with the irradiation system the to-be-harvested algae based on the one or more control signals.

14. A computer program comprising instructions which, when executed by a processor of data processing system of a system as claimed in one of claim 1, causes the processor to perform the steps of:
receiving, from the measurement system a signal indicative of one or more parameters that are relatable to a property of to-be-harvested algae,
determining, based on the one or more parameters as indicated by the signal, a pre-harvest radiation recipe, wherein the pre-harvest radiation recipe is configured to provide radiation comprising a large fraction of blue radiation UV-A radiation or UV-B radiation,
generating one or more control signals for an irradiation system to irradiate the to-be-harvested algae in accordance with the determined pre-harvest radiation recipe,
sending the one or more control signals to the irradiation system.

15. A non-transitory computer-readable storage medium having instructions stored thereon, where when the instructions are executed by a processor of a data processing system cause the processor to perform the method of claim 13.
